(19) United States
(12) Patent Application Publication
(10) Pub. No.: US 2023/0381257 A1
(43) Pub. Date: Nov. 30, 2023

(54) HERBAL COMPOSITION JCM-16021 AS TREATMENT FOR INFLAMMATORY BOWEL DISEASE

(71) Applicants: Hong Kong Baptist University, Hong Kong (CN); University of Macau, Macau (CN)

(72) Inventors: Zhaoxiang BIAN, Hong Kong (CN); Ying WANG, Macau (CN); YiTao WANG, Macau (CN)

(21) Appl. No.: 18/321,856

(22) Filed: May 23, 2023

Related U.S. Application Data
(60) Provisional application No. 63/346,332, filed on May 27, 2022.

(30) Foreign Application Priority Data
Jan. 18, 2023 (CN) 2023100705961

(51) Int. Cl. A61K 36/185; A61K 36/65; A61K 36/575; A61K 36/66; A61K 36/704; A61K 36/284; A61K 36/8994; A61P 1/04

(52) U.S. Cl. CPC A61K 36/185; A61K 36/65; A61K 36/575; A61K 36/66; A61K 36/704; A61K 36/284; A61K 36/8994; A61P 1/04; A61K 2236/15; A61K 2236/55; A61K 2236/33

(57) ABSTRACT

An herbal composition including Fructus Terminaliae Chebulae, Radix Paeoniae Lactiflorae, Cortex Magnoliae Officinalis, Rhizoma Corydalis Yanhusuo, Herba Polygoni Chinensis, Rhizoma Atractylodis Macrocephalae, and Semen coicis Lachryrna-jobi useful for treating diseases, such as inflammatory bowel disease, by increasing regulatory T cells in a subject in need thereof, methods for preparing of use and preparation thereof.

### CROSS-REFERENCE TO RELATED APPLICATIONS

The present application claims priority from China Patent Application No. 202310070596.1, filed on Feb. 7, 2023, which claims priority from U.S. Provisional Patent Application No. 63/346,332 filed on May 27, 2022, which are hereby incorporated by references in their entirety.

### REFERENCE TO SEQUENCE LISTING

The Sequence Listing identified as Sequence_Listing_P25223US00.xml; Size: 6,125 bytes; and Date of Creation: May 15, 2023, filed herewith, is hereby incorporated by reference in its entirety.

### TECHNICAL FIELD

The present disclosure relates to the field of herbal compositions and more particularly relates to an herbal composition and methods of treating inflammatory bowel disease, such as ulcerative colitis, using the herbal composition and methods of preparation thereof.

### BACKGROUND

Inflammatory bowel disease (IBD) is a chronic gastrointestinal inflammatory disease that affects the entire gastrointestinal tract and colon. IBD results in inflammation or destruction of the intestinal wall that can lead to intestinal ulcers and narrowing. The two most common types of IBD are ulcerative colitis and Crohn's disease. For many patients, IBD is hereditary, but no single gene has been shown to cause IBD. Typically, the severity of the disease depends on a combination of genetic abnormalities and any number of microbial influences on the immune system. The most common symptom of IBD is diarrhea. Other symptoms can include abdominal cramps, bloody stools, intestinal obstruction, fever, fluid loss and loss of appetite, extreme weight loss and anemia.

In today's industrialized world, there are over 5 million people with IBD. In Europe, the incidence of ulcerative colitis and Crohn's disease is 0.5% and 1%, respectively. In recent years, there has been a sharp increase in the number of patients with IBD in Asia, particularly in China. The main features of IBD are diarrhea, abscesses, fistulas, abdominal pain and narrowing, all of which have a significant impact on patients' quality of life.

Current treatments for IBD mainly include immunosuppressants and steroids. Neutralizing antibodies against inflammatory cytokines or anti-integrin proteins to block interactions between circulating leukocytes and intestinal blood vessels. The first FDA-approved neutralizing antibody for treating IBD was anti-tumor necrosis factor (TNF-α) (a monoclonal antibody), infliximab (IFLX). IFLX specifically binds to cytoplasmic and membrane-bound TNF-α and subsequently induces apoptotic cell death in cells in an inflamed state. However, long-term use of IFLX can increase clinical remission in patients with IBD. The property of IFLX and many other biologics as exogenous proteins also leads to the production of anti-drug antibodies and an increased risk of malignancy. A five-year study showed that long-term use of humanized monoclonal antibodies against interleukin (IL)-12/IL-23 ustekinumab can alleviate moderate to severe Crohn's disease in patients. Anti-integrin drugs such as vedolizumab also show high maintenance rates for remission. Long-term use of immunosuppressants such as mesalamine increases patient susceptibility to infection. In addition, due to serious side effects, corticosteroids are not recommended for maintaining remission. There is thus an urgent need for more effective therapies with different modes of action and lacking immunogenicity for long-term use in patients with IBD, especially for patients in remission.

### SUMMARY

In view of the above technical problems, on the one hand, this disclosure provides an herbal composition JCM-16021 comprising: Fructus Terminaliae Chebulae, Radix paeoniae Lactiflorae, Cortex Magnoliae Officinalis, Rhizoma Corydalis Yanhusuo, Herba Polygoni Chinensis, Rhizoma Atractylodis Macrocephalae, and Semen coicis Lachryma-jobi.

On the other hand, the present disclosure also relates to the use of the herbal composition JCM-16021 in the preparation of drugs for the treatment of IBD. In certain embodiments, the disease is ulcerative colitis or Crohn's disease. In certain embodiments, the disease is ulcerative colitis.

On the other hand, the present disclosure also relates to a method for preparing a drug comprising the herbal composition JCM-16021.

In a first aspect, provided herein is a method of treating a disease by increasing regulatory T cells in a subject in need thereof, the method comprising: administering a therapeutically effective amount of a herbal composition comprising: Fructus Terminaliae Chebulae, Radix Paeoniae Lactiflorae, Cortex Magnoliae Officinalis, Rhizoma Corydalis Yanhusuo, Herba Polygoni Chinensis, Rhizoma Atractylodis Macrocephalae, and Semen coicis Lachryrna-jobi; and optionally a pharmaceutically acceptable carrier.

In certain embodiments, the herbal composition comprises: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryrna-jobi, 15-21% w/w.

In certain embodiments, the herbal composition comprises: Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis, 18% w/w; Rhizoma Atractylodis Macrocephalae, 18% w/w; and Semen coicis Lachryrna-jobi, 18% w/w.

In certain embodiments, the disease is inflammatory bowel disease.

In certain embodiments, the inflammatory bowel disease is ulcerative colitis or Crohn's disease.

In certain embodiments, the herbal composition is provided in the form of a tablet, a pill, a drop, a capsule, granules, a powder, a suppository, powders for dispersion in water or other liquid for oral administration or for external application to skin or mucous membranes, a patch, an injection, a solution, a suspension, a spray, a wash, a drop, a rub, or an emulsion.

In certain embodiments, the herbal composition is provided in the form of an injection, a solution, a suspension, a spray, a wash, a drop, a rub, or an emulsion.

In certain embodiments, the herbal composition is provided at a concentration of about 0.09-0.18 mg/mL.

In certain embodiments, the pharmaceutically acceptable carrier comprises: a saline solution, a sugar, a gelatin, starch, Ringer's solution, or cellulose.

In certain embodiments, the pH of the pharmaceutically acceptable carrier is 3-11, 5-9, or 7-8.

In certain embodiments, wherein the herbal composition is administered at a dose of 1-4 g/kg body weight or 2 g/kg body weight.

In a second aspect, provided herein is a method for preparing a drug composition comprising an herbal composition, the method comprising: grinding the herbs and an optional pharmaceutically acceptable carrier into a dry powder; adding the dry powder to a water-soluble solvent and incubating at 60° C. for 30 minutes; adding distilled water; and separating the supernatant from debris, wherein the herbal composition comprises:
Fructus Terminaliae Chebulae, about 6-12% w/w;
Radix Paeoniae Lactiflorae, about 11-17% w/w;
Cortex Magnoliae Officinalis, about 6-12% w/w;
Rhizoma Corydalis Yanhusuo, about 11-17% w/w;
Herba Polygoni Chinensis, about 15-21% w/w;
Rhizoma Atractylodis Macrocephalae, about 15-21% w/w; and
Semen coicis Lachryma-jobi, about 15-21% w/w.

In a third aspect, provided herein is an herbal composition comprising: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryrna-jobi, 15-21% w/w.

In a fourth aspect, provided herein is a drug composition comprising an herbal composition described herein and a pharmaceutically acceptable carrier.

In certain embodiments, the herbal composition is prepared by grinding the herbs and an optional pharmaceutically acceptable carrier into a dry powder; adding the dry powder to a water-soluble solvent and incubating at 60° C. for 30 minutes; adding distilled water; centrifuging at room temperature at a speed of from 7,500-12,000 rpm for from minutes and separating the supernatant from debris.

In certain embodiments, the water-soluble solvent comprises polyethylene glycol (15)-hydroxystearate ester.

In certain embodiments, the herbal composition is provided in the form of a tablet, a pill, a drop, a capsule, a granule, a powder, a suppository, a powder for dispersion in water or other liquid for oral administration or for external application to skin or mucous membranes, a patch, an injection, a solution, a suspension, a spray, a wash, a drop, a rub, an emulsion.

In certain embodiments, the drug composition is provided in the form of an injection, a solution, a suspension, a sprays, a wash, a drop, a rub, or an emulsion.

In certain embodiments, the drug composition is provided at a concentration of about 0.09-0.18 mg/mL.

In certain embodiments, drug composition is provided at a concentration of about mg/mL.

In certain embodiments, the pH of the carrier is 3-11, 5-9, or 7-8.

In vivo experimental results show that JCM-16021 exhibits significant therapeutic effects in two mouse models of ulcerative colitis induced by CD4⁺ naive T cell transfer and dextran sulfate sodium. At a treatment dose of 2.0 g/kg body weight, JCM-16021 significantly reduced pathological indicators of ulcerative colitis, promoted proliferation of stem cells in the colonic crypt region and alleviated systemic inflammation in experimental colitis model mice. In addition, JCM-16021 treatment increased the proportion of CD4⁺Foxp3⁺ regulatory T cells (Tregs) in colonic lamina propria lymphocytes, restored gut microbiota composition and increased gut aryl hydrocarbon receptor (AHR) expression. Moreover, the overall therapeutic effect of JCM-16021 was superior to that of IFLX used clinically, especially in promoting proliferation of gut epithelial cells and CD4⁺Foxp3⁺ T_{reg} cells.

Unless otherwise specified or clearly inconsistent with the context in this invention, all methods described herein can be performed in any suitable order according to the understanding of those skilled in the art.

All patents, patent applications and references cited herein are incorporated herein by reference in their entirety as if each individual publication were specifically and individually indicated to be incorporated by reference. If there is a conflict between this invention and any document provided herein, the contents of this invention shall prevail.

### BRIEF DESCRIPTION OF DRAWINGS

FIG. 1 shows the therapeutic effect of JCM-16021 on ulcerative colitis induced by CD4⁺ naive T cell transfer. (A) Rag 1^{-/-} immunodeficient mice aged 6-8 weeks were intraperitoneally injected with CD4⁺ naive T cells at 2×10⁵/mouse. Treatment with JCM-16021 (oral administration twice daily) began three weeks after injection with IFLX as a positive control drug (intravenous injection once weekly) and sham control group received oral saline. (B) Changes in body weight during treatment for each group of mice; arrows indicate start of JCM-16021 or IFLX treatment. (C) Fecal occult blood index. (D) Ulcerative colitis pathological index. (E) Hematoxylin-eosin staining (H&E staining), periodic acid-Schiff staining (PAS staining), Claudin and NF-κB p65 immunohistochemistry results for mouse colon tissue sections. (B)-(D) Values are expressed as mean±standard error (SEM). Scale bar=100 μm. Results from three groups of experiments (*, p<0.05; more than 5 mice per group).

FIG. 2 shows JCM-16021 treatment suppressed inflammation in naïve T cell transfer-induced colitis. Representative immunofluorescent staining images of (A) NF-κB p65 or c-Rel costained with Iba1 in colon sections of all treatment groups. Hoechst labels the nucleus. Scale bar=100 μm. Results are representative from three separate experiments (n≥5 per group). Relative mRNA expression levels of (B) Tnf-α, and (C) Mcp-1 in the colon from mice treated as indicated. Data are presented as mean±S.E.M. (**, p<0.01).

FIG. 3 shows PCNA and CD44 immunofluorescence staining results for colon tissue from Rag 1^{-/-} immunodeficient mice with ulcerative colitis induced by CD4⁺ naive T cell transfer treated with JCM-16021. (A) Immunofluorescence detection of AHR in small intestine tissue from mice treated with JCM-16021 for ulcerative colitis induced by CD4⁺ naive T cell transfer. (B) Immunofluorescent images of AHR and pan-keratin expression in the colon. (C) Immunofluorescent images of AHR and MUC2 expression in the colon. Images are representative from three separate experiments. Scale bar=100 μm (more than 5 mice per group).

FIG. 4 shows the proportion of regulatory T cells among immune cells in the colon lamina propria from Rag 1^{-/-} immunodeficient mice with ulcerative colitis induced by CD4⁺ naive T cell transfer treated with JCM-16021. (A) Schematic diagram of flow cytometry analysis. (B) Proportion of CD4⁺Foxp3⁺ cells among all CD4⁺ cells. (B) Values are expressed as mean±standard error (SEM) (*, p<0.05; more than 5 mice per group).

FIG. 5 shows relative abundance at species level for gut microbiota from Rag 1^{-/-} immunodeficient mice with ulcerative colitis induced by CD4⁺ naive T cell transfer treated with JCM-16021. (A) Bacteroidales S24-7; (B) Prevotellaceae; (C) Rikenellaceae; (D) Helicobacteraceae; (E) Deferribacteraceae; (F) Rhodospirillaceae; (G) Clostridiaceae; (H) Christensenellaceae species. Values are expressed as mean±standard error (SEM) (*, p<0.05; **, p<0.01; ***, p<0.001; n.s., not significant).

FIG. 6 shows therapeutic effect of JCM-16021 on long-term dextran sulfate sodium-induced ulcerative colitis mouse model. (A) Changes in body weight during treatment for each group of mice. (B) Colon length for each group of mice. (C) Fecal occult blood index. (D) Colitis pathological index. Values are expressed as mean±standard error (SEM). (*, p<0.05; **, p<0.01; ***, p<0.005; n.s., not significant).

FIG. 7 shows changes in pathological indicators of colon tissue from mice with 2% dextran sulfate sodium-induced ulcerative colitis treated with JCM-16021. (A) Hematoxylin-eosin staining and periodic acid-Schiff staining results for mouse colon tissue sections; scale bar=100 μm (B) PCNA and CD44 immunofluorescence staining results for colon tissue; results from three replicate immunofluorescence experiments (n≥5 per group).

FIG. 8 shows levels of pro-inflammatory cytokines (A) Ifn-γ, (B) Tnf-α and chemokines, (C) Mcp-1, (D) Gm-csf in serum from mice with 2% dextran sulfate sodium-induced ulcerative colitis treated with JCM-16021. Values are expressed as mean±standard error (SEM). (*, p<0.05; **, p<0.01; **, p<0.005; n.s., not significant).

FIG. 9 shows that treatment with JCM-16021 at 2.0 g/kg induced expression of AHR and MUC2 in DSS-induced colitis. (A) Immunofluorescent images of AHR and pan-keratin expression in the colon. (B) Immunofluorescent images of AHR and MUC2 expression in the colon. Images are representative from three separate experiments. Scale bar=100 μm (n≥5 per group).

FIG. 10 shows the quantitative results of the percentage of CD4⁺Foxp3⁺ cells in total CD4⁺ cells in (A) lamia propria lymphocytes (LPMC), (B) mesenteric lymph nodes (MLN), and (C) spleen of JCM-16021-treated colitis mice induced by DSS. Data are presented as mean±standard error (SEM) (**, p<0.01; n≥5 per group).

FIG. 11 shows relative abundance at species level for gut microbiota from mice with long-term dextran sulfate sodium-induced ulcerative colitis treated with JCM-16021. (A) Dubosiella; (B) Fissicatena; (C) Clostridium; (D) Dubosiella; (E) Lachnospiraceae bacterium. Values are expressed as mean±standard error (SEM). (*, p<0.05; **, p<0.01; ***, p<0.005).

### DETAILED DESCRIPTION

### Definitions

Unless otherwise specified, all technical and scientific terms used herein have the same meaning as commonly understood by those skilled in the art to which this invention pertains, but if there is a conflict, the definitions in this specification shall prevail.

As used in this specification and the appended claims, the singular forms “a,” “an,” and “the” include plural referents unless the context clearly dictates otherwise.

All ranges disclosed herein that relate to amounts of components or process conditions are inclusive of endpoints and independently combinable. Since these ranges are continuous, they include every value between the minimum and maximum values. It should also be understood that any numerical range disclosed herein is intended to include all subranges within that range and all specific embodiments within that range.

When this invention refers to physical properties such as molecular weight or chemical properties by range definition, it should include all combinations and subcombinations thereof as well as specific embodiments within it. The term “comprising” (and related terms such as “containing” or “including” or “having” or “including”) includes such embodiments as, for example, any combination of substances, compositions, methods or processes that are “composed of the described features” or “essentially composed of the described features.”

The term “and/or” used herein should be understood to mean either one or both of the associated components “either one or both,” i.e., the components coexist in some cases and exist separately in others. Multiple components listed with “and/or” should be understood in the same way as “one or more” associated components. In addition to components specifically identified by an “and/or” clause, other components may optionally be present whether related or unrelated to those specifically identified components. Thus, for example, reference to “A and/or B,” when used to connect open-ended text such as “comprising,” may refer only to A (optionally including components other than B) in one embodiment; only to B (optionally including components other than A) in another embodiment; or to A and B (optionally including other components) in yet another embodiment.

The term “administering,” “giving,” or “administering” means implanting, absorbing, ingesting, injecting, inhaling, or otherwise introducing into a subject the herbal composition, extract thereof or drug composition described herein.

The terms “treating” and “treating” mean reversing, alleviating, delaying onset or inhibiting progression of the disease described herein. In some embodiments, treatment may be administered after the disease has occurred or one or more signs or symptoms have been observed. In other embodiments, treatment may be administered in the absence of signs or symptoms of the disease. For example, treatment may be administered prior to symptom onset to susceptible subjects (e.g., based on symptom history and/or exposure to pathogens) to delay or prevent disease onset. Treatment may also continue after symptoms have disappeared, for example to delay and/or prevent recurrence.

The terms “condition,” “disease,” and “disorder” may be used interchangeably.

An “effective amount” of an herb described herein is an amount sufficient to cause a desired biological response. An effective amount of a compound described herein may vary depending on factors such as: expected biological endpoint; pharmacokinetics of compound; disease being treated; route of administration; age and health status of subject. In some embodiments, an effective amount is a therapeutically effective amount. In some embodiments, an effective amount is a prophylactically effective amount. In some embodiments, an effective amount is an amount of an herbal composition or herb or drug described herein in a single dose. In some embodiments, an effective amount is a combined amount of an herbal composition or drug described herein in multiple doses.

A “therapeutically effective amount” of a compound described herein is an amount sufficient to provide therapeutic benefit for treating a disease or delaying or minimizing one or more symptoms associated with that disease. A therapeutically effective amount of a therapeutic agent refers to an amount alone or in combination with other therapies that provides therapeutic benefit for treating that disease. The term “therapeutically effective amount” may include amounts that improve overall therapy; alleviate or eliminate symptoms; signs; causes; and/or enhance efficacy of another therapeutic agent.

The term “pharmaceutically acceptable” indicates that the substance or composition is chemically and/or toxicologically compatible with other ingredients comprising the formulation and/or with the subject being treated.

A subject being administered a drug composition described herein includes but is not limited to: humans (i.e., males or females of any age group such as pediatric subjects (e.g., infants; children; adolescents) or adult subjects (e.g., young adults; middle-aged adults; older adults)) and/or non-human animals such as mammals such as primates (e.g., macaques; rhesus monkeys); cattle; pigs; horses; sheep; goats; rodents; cats; and/or dogs.

In certain embodiments, the subject is a human. In some embodiments, the subject is a non-human animal. The terms “human,” “patient,” “individual in need,” and “subject” may be used interchangeably herein.

Unless otherwise defined, all technical and scientific terms used herein have the same meaning as commonly understood by those skilled in the art to which this invention pertains. The terms used in this specification are for the purpose of describing particular embodiments only and are not intended to limit the invention.

In order to facilitate understanding of this invention, a more comprehensive description of this invention will be provided below. However, this invention can be implemented in different forms and is not limited to the embodiments described herein. The purpose of providing these embodiments is to make the disclosure of the content of this invention more thorough and comprehensive.

The gut microbiota and AHR axis are important regulatory factors in the pathogenesis of IBD, such as ulcerative colitis. One major reason for limited treatment strategies is that the pathogenic factors that trigger IBD are very complex and largely unresolved. Commensal bacteria in the gut are the foundation of gut homeostasis and metabolism. Loss of balance within the gut microbiota, or dysbiosis, directly activates immune responses within the gut by initiating innate immune responses. Dysbiosis also leads to metabolic impairment, including biosynthesis of 5-hydroxytryptamine (5-HT). Microbiota-mediated tryptophan metabolites, such as indole-3-aldehyde, directly activate AHR-dependent IL-22 transcription as ligands. IL-22-dependent mucosal immune responses thus alter the composition of microbial communities and gut immune responses. Therefore, the gut microbiota-AHR axis is an important therapeutic target for IBD treatment.

T_{reg} cell therapy is promising in IBD treatment. In IBD, chronic intestinal inflammation leads to functional abnormalities of CD4⁺Foxp3⁺ T_{reg} cells in vivo. In turn, impaired T_{reg} cells impair robust tissue repair and regeneration, further amplifying the inflammatory response. Therefore, restoring T_{reg} cell inhibitory function in the context of intestinal inflammation is very promising in treating inflammatory and autoimmune diseases including IBD. However, manipulating T_{reg} cell numbers or abundance to achieve immune response homeostasis is very challenging. Only a few compounds have shown activity in altering T_{reg}/T helper cell (Tₕ) 17 balance or activating T_{reg} cell immune inhibitory function. Alternatively, methods using chimeric antigen receptor co-receptors designed on cell surfaces have been used to direct Tre g cells to target antigens. Research on manipulating T_{reg} cells in inflammatory and autoimmune diseases is still in its infancy to evaluate the efficacy of reinfusing autologous ex vivo expanded T_{reg} cells into donor patients. There remains a critical gap between bioengineered manipulation of Tre g cells and clinically approved IBD therapies.

Herbal composition JCM-16021 shows significant efficacy for treating ulcerative colitis via a new mechanism of action. Herbal composition JCM-16021 is modified based on Tongxie Yaofang formula, which has been used in China since the 12^{th} century to treat abdominal pain and diarrhea. JCM-16021 alleviated visceral hyperalgesia in rats with post-inflammatory (PI)-IBS model induced by trinitrobenzenesulfonic acid. Treatment with JCM-16021 significantly improved intestinal chromaffin cell numbers, intestinal 5-HT content, tryptophan hydroxylase expression and mechanically stimulated 5-HT release. In addition, JCM-16021 reduced levels of Th₁ cell-associated pro-inflammatory cytokine TNF-α in PI-IBS rats.

The data presented herein demonstrates that JCM-16021 exhibits significant efficacy in both ulcerative colitis induced by naive CD4⁺ T cell transfer in Rag 1^{-/-} immunodeficient mice and experimental colitis induced by dextran sulfate sodium (DSS) in C57BL/6J mice. In experimental colitis models, JCM-16021 at a dose of 2.0 g/kg significantly induced recovery of pathological indices of ulcerative colitis, promoted proliferation of stem cells in the colonic crypt region and alleviated systemic inflammation. In addition, JCM-16021 treatment promoted the proportion of CD4⁺Foxp3⁺ T_{reg} cells in lamina propria lymphocytes (LPMC), restored gut microbiota composition and gut expression of AHR. The findings disclosed herein provide key evidence that the overall efficacy of JCM-16021 is superior to that of IFLX used clinically, especially in promoting gut regeneration and CD4⁺Foxp3⁺ T_{reg} cell populations. In certain embodiments, the effective dose of JCM-16021 in experimental ulcerative colitis models is 1-4 g/kg body weight, preferably 2 g/kg body weight.

Herbal Composition and Drug Composition

In one aspect of the present disclosure, provided herein is an herbal composition JCM-16021 (also referred to herein as JCM-16021), which comprises the following components: Fructus Terminaliae Chebulae, Radix Paeoniae Lactiflorae, Cortex Magnoliae Officinalis, Rhizoma Corydalis Yanhusuo, Herba Polygoni Chinensis, Rhizoma Atractylodis Macrocephalae, and Semen coicis Lachryma-jobi. In certain embodiments, the herbal composition comprises the following components (herbs): Fructus Terminaliae Chebulae, Radix Paeoniae Lactiflorae, Cortex Magnoliae Officinalis, Rhizoma Corydalis Yanhusuo, Herba Polygoni Chinensis, Rhizoma Atractylodis Macrocephalae, and Semen coicis Lachryma-jobi.

In certain embodiments, the components of the above herbal composition have the following weight ratios: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryma-jobi, 15-21% w/w.

In certain embodiments, the weight ratio of Fructus Terminaliae Chebulae in the above herbal composition is: 6-11% w/w; 6-10% w/w; 6-9% w/w; 6-8% w/w; 6-7% w/w; or 9% w/w.

In certain embodiments, the weight ratio of Radix Paeoniae Lactiflorae in the above herbal composition is: 11-16% w/w; 11-15% w/w; 11-14% w/w; 11-13% w/w; 11-12% w/w; or 14% w/w.

In certain embodiments, the weight ratio of Cortex Magnoliae Officinalis in the above herbal composition is: 6-11% w/w; 6-10% w/w; 6-9% w/w; 6-8% w/w; 6-7% w/w; or 9% w/w.

In certain embodiments, the weight ratio of Rhizoma Corydalis Yanhusuo in the above herbal composition is: 11-16% w/w; 11-15% w/w; 11-14% w/w; 11-13% w/w; 11-12% w/w; or 14% w/w.

In certain embodiments, the weight ratio of Herba Polygoni Chinensis in the above herbal composition is: 15-20% w/w; 15-19% w/w; 15-18% w/w; 15-17% w/w; 15-16% w/w; or 18% w/w.

In certain embodiments, the weight ratio of Rhizoma Atractylodis Macrocephalae in the above herbal composition is: 15-20% w/w; 15-19% w/w; 15-18% w/w; 15-17% w/w; 15-16% w/w; or 18% w/w.

In certain embodiments, the weight ratio of Semen coicis Lachryma-jobi in the above herbal composition is: 15-20% w/w; 15-19% w/w; 15-18% w/w; 15-17% w/w; 15-16% w/w; or 18% w/w.

In certain embodiments, the composition comprises: Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis, 18% w/w; Rhizoma Atractylodis Macrocephalae, 18% w/w; and Semen coicis Lachryma-jobi, 18% w/w.

In certain embodiments, the herbal composition is used to promote an increase in regulatory T cells in an individual in need. Specifically, in some embodiments, the herbal composition is used to treat IBD in an individual in need. In other embodiments, the IBD includes ulcerative colitis and Crohn's disease. In other embodiments, the IBD is ulcerative colitis.

On the other hand, this disclosure also relates to an extract of the above herbal composition. In certain embodiments, the extract is an aqueous solvent extract. In certain embodiments, the extract comprises polyethylene glycol (15)-hydroxystearate. In certain embodiments, the extract is a distilled water extract. In certain embodiments, the extract is a physiological saline extract.

On the other hand, this disclosure also relates to a drug composition comprising a pharmaceutically acceptable carrier and material or chemical substance from the following plant species: Fructus Terminaliae Chebulae, Radix Paeoniae Lactiflorae, Cortex Magnoliae Officinalis, Rhizoma Corydalis Yanhusuo, Herba Polygoni Chinensis, Rhizoma Atractylodis Macrocephalae, and Semen coicis Lachryma-jobi. In certain embodiments, the drug composition consists essentially of the following components: Fructus Terminaliae Chebulae, Radix Paeoniae Lactiflorae, Cortex Magnoliae Officinalis, Rhizoma Corydalis Yanhusuo, Herba Polygoni Chinensis, Rhizoma Atractylodis Macrocephalae, and Semen coicis Lachryma-jobi. In certain embodiments, the drug composition is used to treat IBD in mammals. In certain embodiments, the drug composition is used to treat ulcerative colitis in mammals. In certain embodiments, the drug composition is used to treat Crohn's disease in mammals. In certain embodiments, the drug composition is used as an immunotherapy to promote an increase in regulatory T cells for treating IBD, such as ulcerative colitis.

On the other hand, this disclosure also relates to a drug composition comprising a pharmaceutically acceptable carrier and the above herbal composition. In certain embodiments, the pharmaceutically acceptable carrier includes physiological saline; sugars; gelatin; starch; Ringer's solution; and/or cellulose.

In certain embodiments, the pH of the pharmaceutically acceptable carrier is 3-11. In certain embodiments, the pH of the pharmaceutically acceptable carrier is 4-11. In certain embodiments, the pH of the pharmaceutically acceptable carrier is 4-10. In certain embodiments, the pH of the pharmaceutically acceptable carrier is 5-10. In certain embodiments, the pH of the pharmaceutically acceptable carrier is 5-9. In certain embodiments, the pH of the pharmaceutically acceptable carrier is 6-8. In certain embodiments, the pH of the pharmaceutically acceptable carrier is 7-8. In certain embodiments, the pH of the pharmaceutically acceptable carrier is 5-9 or 7-8.

In certain embodiments, the herbal composition is provided in an effective amount in the drug. In certain embodiments, the effective amount is a therapeutically effective amount. In certain embodiments, the effective amount is an amount effective for treating IBD in a subject in need thereof. In certain embodiments, the effective amount is an amount effective for treating ulcerative colitis in a subject in need thereof. In certain embodiments, the effective amount is an amount effective for treating Crohn's disease in a subject in need thereof. In certain embodiments, the effective amount is an amount effective for promoting an increase in regulatory T cells in a subject thereof.

In certain embodiments, the drug composition is administered at a dose of about 1-4 g/kg (subject body weight). In certain embodiments, the drug composition is administered at a dose of about 2 g/kg (subject body weight).

In certain embodiments, the drug composition may be administered at specific doses multiple times at intervals, such as twice daily, once daily, six times weekly, five times weekly or less.

In certain embodiments, the drug composition may be administered by inhalation; intranasal; intravenous; intramuscular; subcutaneous; topical; oral; transdermal; intraperitoneal; or oral administration.

In certain embodiments, the drug composition may be used in combination with other drugs, e.g., therapeutically effective amounts of other drugs to treat IBD.

In certain embodiments, the drug composition may be used in combination with other drugs, e.g., therapeutically effective amounts of other drugs to treat ulcerative colitis.

In certain embodiments, the drug composition may be used in combination with other drugs, e.g., therapeutically effective amounts of other drugs to treat Crohn's disease.

In certain embodiments, the drug composition is provided in the form of tablets; pills; drops; capsules; granules; powders; suppositories; powders; ointments; patches; injections; solutions; suspensions; sprays; washes; drops; rubs; emulsions. The formulation can conveniently be presented in unit dosage form and can be prepared by any method known in the art of pharmacy. Techniques and formulations are generally found in Remington's Pharmaceutical Sciences 18th Edition (1995) Mack Publishing Co., Easton, PA. These methods include steps of combining active ingredients with one or more auxiliary components comprising a carrier. Typically, the drug composition is prepared by uniformly and intimately combining active ingredients with a liquid carrier or finely divided solid carrier or both and then shaping the product if necessary.

In certain embodiments, when the pharmaceutical composition is prepared as a liquid dosage form, the pharmaceutical composition is provided at a concentration of about 0.09-0.18 mg/mL. In certain embodiments, the pharmaceutical composition is provided at a concentration of about 0.10-0.18 mg/mL. In certain embodiments, the pharmaceutical composition is provided at a concentration of about 0.11-0.18 mg/mL. In certain embodiments, the pharmaceutical composition is provided at a concentration of about 0.12-0.18 mg/mL. In certain embodiments, the pharmaceutical composition is provided at a concentration of about 0.13-0.18 mg/mL. In certain embodiments, the pharmaceutical composition is provided at a concentration of about 0.14-0.18 mg/mL. In certain embodiments, the pharmaceutical composition is provided at a concentration of about 0.15-0.18 mg/mL. In certain embodiments, the pharmaceutical composition is provided at a concentration of about 0.16-0.18 mg/mL. In certain embodiments, the pharmaceutical composition is provided at a concentration of about 0.17-0.18 mg/mL. In certain embodiments, the pharmaceutical composition is provided at a concentration of about 0.09 mg/mL. In certain embodiments, the pharmaceutical composition is provided at a concentration of about 0.10 mg/mL. In certain embodiments, the pharmaceutical composition is provided at a concentration of about 0.11 mg/mL. In certain embodiments, the pharmaceutical composition is provided at a concentration of about 0.12 mg/mL. In certain embodiments, the pharmaceutical composition is provided at a concentration of about 0.13 mg/mL. In certain embodiments, the pharmaceutical composition is provided at a concentration of about 0.14 mg/mL. In certain embodiments, the pharmaceutical composition is provided at a concentration of about 0.15 mg/mL. In certain embodiments, the pharmaceutical composition is provided at a concentration of about 0.16 mg/mL. In certain embodiments, the pharmaceutical composition is provided at a concentration of about 0.17 mg/mL. In certain embodiments, the pharmaceutical composition is provided at a concentration of about 0.18 mg/mL. In certain preferred embodiments, the pharmaceutical composition is provided at a concentration of 0.18 mg/mL.

A pharmaceutically acceptable carrier (or therapeutically acceptable carrier) is well known to those skilled in the art and includes substances such as carbohydrates, waxes, water-soluble and/or swellable polymers, hydrophilic or hydrophobic substances, gelatin, oils, solvents, water, etc. The specific carrier, diluent or excipient used depends on the mode and target of application of the compound described herein. Solvents are generally chosen based on those considered safe for administration to mammals by those skilled in the art (GRAS). Typically, safe solvents are non-toxic aqueous solvents such as water and other non-toxic solvents soluble or miscible in water. Suitable aqueous solvents include water; ethanol; propylene glycol; polyethylene glycol (e.g., PEG400; PEG300); etc. and mixtures thereof. The pharmaceutically acceptable carrier may also include one or more buffers; formulating agents; diluents; stabilizers; surfactants; wetting agents; lubricants; emulsifiers; suspending agents; preservatives; antioxidants; light protectants; flow aids; processing aids; colorants; sweeteners; flavorings; flavor correctants and other known additives to provide a pleasing appearance to the drug (i.e., the herbal composition of this invention or a drug containing it) or to facilitate the manufacture of the drug.

Exemplary diluents include calcium carbonate, sodium carbonate, calcium phosphate, dicalcium phosphate, calcium sulfate, calcium hydrogen phosphate, sodium phosphate, sugars such as sucrose, mannitol, sorbitol or xylitol, cellulose, microcrystalline cellulose, kaolin, mannose alcohol, sorbitol alcohol, inositol, sodium chloride, dry starches such as corn starch and powdered sugar and mixtures thereof.

Exemplary granulating and/or dispersing agents include potato starches such as corn starch and cassava starches such as sodium starch glycolate (Explotab), clays such as alginates such as sodium alginate (Kelgin), guar gum (Jaguar), carob bean gum (Locust bean gum), ghatti gum (ghatti gum), mucilage of isapol husks (mucilage of isapol husks), carboxymethyl cellulose (CMC), methyl cellulose (MC), ethyl cellulose (EC), hydroxyethyl cellulose (HEC), hydroxypropyl cellulose (HPC), hydroxypropyl methyl cellulose (HPMC), microcrystalline cellulose (MCC), cellulose acetate (CA), poly(vinylpyrrolidone) (PVP), magnesium aluminum silicate (Veegum®) and larch arabogalactan)), alginates such as sodium alginate (Kelgin®), polyethylene oxide (PEO), polyethylene glycol (PEG), inorganic calcium salts such as silicates such as poly(methyl methacrylate) (PMMA), waxes such as water such as ethanol and/or mixtures thereof.

Exemplary binders include starches such as corn starch and starch paste); gelatin; sugars such as sucrose; glucose; dextrose; maltodextrin; molasses; lactose; lactitol; mannitol alcohol etc.; natural and synthetic gums such as gum arabic; sodium alginate; carrageenan extract; panwar gum (panwar rum); Indian gum (ghatti gum); mucilage of isapol husks); carboxymethyl cellulose (CMC); methyl cellulose (MC); ethyl cellulose (EC); hydroxyethyl cellulose (HEC); hydroxypropyl cellulose (HPC); hydroxypropyl methyl cellulose (HPMC); microcrystalline cellulose (MCC); cellulose acetate (CA); poly(vinylpyrrolidone) (PVP); magnesium aluminum silicate (Veegum®) and larch arabogalactan)), alginate salts such as sodium alginate (Kelgin®), polyethylene oxide (PEO), polyethylene glycol (PEG), inorganic calcium salts such as silicates such as poly(methyl methacrylate)(PMMA), waxes such as water such as ethanol and/or mixtures thereof.

Exemplary buffers include citrate salt buffer solutions, acetate salt buffer solutions: Phosphate buffer solutions, ammonium chloride, calcium carbonate, calcium chloride, calcium citrate, calcium gluconolactate, calcium glucoheptonate, calcium gluconate, D-gluconic acid, calcium glycerophosphate, calcium lactate, propionic acid, calcium propionate, valeric acid, calcium hydrogen phosphate, phosphoric acid, calcium phosphate, basic calcium phosphate, potassium acetate, potassium chloride, potassium gluconate, potassium mixtures, dipotassium hydrogen phosphate, potassium dihydrogen phosphate, potassium phosphate mixtures, sodium acetate, sodium bicarbonate, sodium chloride, sodium citrate, sodium lactate, disodium hydrogen phosphate, sodium dihydrogen phosphate, sodium phosphate mixtures; aminobutanol; magnesium hydroxide; aluminum hydroxide; alginic acid; pyrogen-free water; isotonic saline; Ringer's solution; ethanol; and mixtures thereof.

Exemplary lubricants include magnesium stearate, calcium stearate, stearic acid, silicon dioxide, talc, maltodextrin, glyceryl behenate (glyceryl behenate), hydrogenated vegetable oil, polyethylene glycol, sodium benzoate, sodium acetate, sodium chloride, leucine, magnesium lauryl sulfate, sodium lauryl sulfate and mixtures thereof.

Liquid dosage forms for oral and enteral administration include pharmaceutically acceptable emulsions; microemulsions; solutions; suspensions; syrups and elixirs. In addition to the active agent(s), the liquid dosage forms may contain inert diluents commonly used in the art such as water; physiological saline or other solvents; solubilizers and emulsifiers such as ethanol; isopropanol; ethyl carbonate; ethyl acetate; benzyl alcohol; benzyl benzoate; propylene glycol; 1,3-butylene glycol; dimethylformamide; oils (e.g., cottonseed oil; peanut oil; corn oil; germ oil; olive oil; castor oil and sesame oil); glycerin; tetrahydrofurfuryl alcohol; polyethylene glycols and fatty acid esters of sorbitan anhydride and mixtures thereof. In addition to inert diluents, oral compositions may contain adjuvants such as wetting agents; emulsifiers and suspending agents; sweeteners; flavorings and perfumes. In some embodiments for enteral administration, the conjugates of this invention are mixed with solubilizers such as Cremophor®; alcohols; oils; modified oils; diols; polyol esters; cyclodextrins; polymers and combinations thereof.

Injectable preparations can be configured using suitable dispersing or wetting agents and suspending agents according to known techniques. For example, injectable sterile aqueous or oily suspensions. Injectable sterile preparations may be injectable sterile solutions, suspensions or emulsions in non-toxic enteral acceptable diluents or solvents such as in 1,3-butylene glycol solution. Acceptable media and solvents that can be used include water, physiological saline, Ringer's solution, U.S.P. and isotonic sodium chloride solution. In addition, sterile non-volatile oils are commonly used as solvents or suspending media. For this purpose any mild non-volatile oil may be used including synthetic mono- or di-glycerides. In addition fatty acids such as oleic acid are used in the preparation of injectable.

Solid dosage forms for oral administration include capsules, tablets, pills, powders and granules. In these solid dosage forms the active ingredient is mixed with at least one inert pharmaceutically acceptable excipient or carrier such as sodium citrate or dicalcium phosphate and/or one or more of the following substances: a) fillers or expanders such as starches such as lactose sucrose glucose mannitol silicates b) binders such as carboxymethyl cellulose alginate salts gelatin polyvinylpyrrolidone sucrose gum arabic c) humectants such as glycerin d) disintegrants such as agar calcium carbonate potato or tapioca starch alginate some silicates sodium carbonate e) solution retardants such as paraffin f) absorption enhancers such as quaternary ammonium compounds g) wetting agents such as monostearin spermaceti ester and monoglyceride h) absorbents such as kaolin and bentonite i) lubricants such as talc calcium stearate magnesium stearate solid polyethylene glycol sodium lauryl sulfate and mixtures thereof. In the case of capsules tablets and pills the dosage form may contain buffers.

In another aspect of the present disclosure, provided is the use of the above herbal composition or drug composition in the preparation of a drug for treating IBD in a subject in need. In certain embodiments, the IBD includes ulcerative colitis and Crohn's disease. In certain embodiments, the IBD is ulcerative colitis. In certain embodiments, the herbal composition or drug composition is administered at a therapeutically effective amount. In certain embodiments, the herbal composition or drug composition is administered with a pharmaceutically acceptable carrier.

In certain embodiments, the present invention also relates to the use of the above herbal composition or drug composition in the preparation of a drug for promoting an increase in regulatory T cells in a subject in need thereof.

In certain embodiments, the present invention also relates to the use of the above herbal composition or drug composition in the preparation of a drug for increasing the proportion of CD4⁺Foxp3⁺ T_{reg} cells in a subject in need thereof.

In certain embodiments, the present invention also relates to the use of the above herbal composition or drug composition in the preparation of a drug for restoring the composition of gut microbiota and restoring expression of AHR in a subject in need thereof.

In another aspect of the present disclosure, the present disclosure relates to the use of the above herbal formula or drug composition in preparing a drug for treating disease in mammals. In certain embodiments, said mammal is human.

Another aspect of this disclosure provides a method for preparing a drug containing said herbal composition comprising: grinding said herbal composition into dry powder; adding said dry powder to polyethylene glycol (15)-hydroxystearate and incubating at 60° C. for 30 minutes; making up volume with distilled water; centrifuging at 7,500-12,000 rpm (preferably 120,000 rpm) at room temperature for 10 minutes to separate supernatant from debris; and optionally storing supernatant at 4° C.

On another hand, this disclosure also relates to following embodiments: Embodiment 1. An herbal composition characterized by comprising components having following weight ratios: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryma-jobi, 15-21% w/w.

Embodiment 2. An herbal composition characterized by comprising components having following weight ratios: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryma-jobi, 15-21% w/w.

Embodiment 3. An herbal composition according to any one of Embodiments 1 or 2 characterized by components having following weight ratios: Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis 18% w/w; Rhizoma Atractylodis Macrocephalae, 18% w/w; and Semen coicis Lachryma-jobi, 18% w/w.

Embodiment 4. A drug composition characterized by comprising an herbal composition according to any one of Embodiments 1-3 and a pharmaceutically acceptable carrier.

Embodiment 5. A drug composition according to Embodiment 4, wherein the drug composition is prepared by the following method: grinding the herbal composition and any selected pharmaceutically acceptable carrier into a dry powder; adding the dry powder to a water-soluble solvent and incubating at 60° C. for 30 minutes; making up volume with distilled water; centrifuging at 7,500-12,000 rpm (preferably 12,000 rpm) at room temperature for 10 minutes to separate supernatant from debris; and optionally storing supernatant at 4° C.

Embodiment 6. A drug composition according to Embodiment 5, wherein the water-soluble solvent is selected from polyethylene glycol (15)-hydroxystearate, distilled water and physiological saline.

Embodiment 7. A drug composition according to any one of Embodiments 4-6, wherein the drug composition is provided in the form of tablets, pills, drops, capsules, granules, powders, suppositories, powders, ointments, patches, injections, solutions, suspensions, sprays, washes, drops, rubs or emulsions.

Embodiment 8. A drug composition according to Embodiment 7, wherein the drug composition is provided in the form of injections; solutions; suspensions; sprays; washes; drops; rubs or emulsions.

Embodiment 9. A drug composition according to Embodiment 8, wherein the drug composition is provided at a concentration of about 0.09-0.18 mg/mL.

Embodiment 10. A drug composition according to Embodiment 9, wherein the drug composition is provided at a concentration of about 0.18 mg/mL.

Embodiment 11. A drug composition according to any one of Embodiments 4-10, wherein the pharmaceutically acceptable carrier includes: physiological saline; sugars; gelatin; starches; Ringer's solution; or cellulose.

Embodiment 12. A drug composition according to any one of Embodiments 4-11, wherein the pH of the carrier is from 3-11; from 5-9; or from 7-8.

Embodiment 13. The use of an herbal composition according to any one of Embodiments 1-3 or a drug composition according to any one of Embodiments 4-12 in preparing a drug for treating IBD in a subject in need thereof.

Embodiment 14. The use according to Embodiment 13 wherein said IBD includes ulcerative colitis and Crohn's disease; or said IBD is ulcerative colitis.

Embodiment 15. The herbal composition according to any one of embodiments 1-3 or the pharmaceutical composition according to any one of embodiments 4-12 in preparation for promoting the increase of Tre g cells in an individual in need thereof use in medicines.

Embodiment 16. The use of an herbal composition according to any one of Embodiments 1-3 or a drug composition according to any one of Embodiments 4-12 in preparing a drug for increasing the proportion of CD4⁺Foxp3⁺ T_{reg} cells in a subject in need thereof.

Embodiment 17. The use of an herbal composition according to any one of Embodiments 1-3 or a drug composition according to any one of Embodiments 4-12 in preparing a drug for restoring the composition of gut microbiota and restoring expression of AHR in a subject in need thereof.

Embodiment 18. The use according to Embodiments 13-17, wherein said subject in need is a mammal; or said mammal is human.

Embodiment 19. A method for preparing a drug composition containing an herbal composition comprising: grinding said herbal composition and any selected pharmaceutically acceptable carrier into dry powder; adding said dry powder to a water-soluble solvent (e.g., selected from polyethylene glycol (15)-hydroxystearate, distilled water and physiological saline) and incubating at 60° C. for 30 minutes; making up volume with distilled water; centrifuging at 7,500-12,000 rpm (preferably 12,000 rpm) at room temperature for 10 minutes to separate supernatant from debris; and optionally storing supernatant at 4° C.;
wherein said herbal composition comprises components having following weight ratios:
   Fructus Terminaliae Chebulae, 6-12% w/w;
   Radix Paeoniae Lactiflorae, 11-17% w/w;
   Cortex Magnoliae Officinalis, 6-12% w/w;
   Rhizoma Corydalis Yanhusuo, 11-17% w/w;
   Herba Polygoni Chinensis, 15-21% w/w;
   Rhizoma Atractylodis Macrocephalae, 15-21% w/w; and
   Semen coicis Lachryma-jobi, 15-21% w/w,
   and in certain embodiments, said herbal composition comprises components having following weight ratios:
Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis, 18% w/w; Rhizoma Atractylodis Macrocephalae, 18% w/w; and Semen coicis Lachryma-jobi, 18% w/w.

On another hand this disclosure also relates to following embodiments:

Embodiment 1. The use of an herbal composition in preparing a drug for treating disease by promoting an increase in regulatory T cells comprising following components: Fructus Terminaliae Chebulae, Radix Paeoniae Lactiflorae, Cortex Magnoliae Officinalis, Rhizoma Corydalis Yanhusuo, Herba Polygoni Chinensis, Rhizoma Atractylodis Macrocephalae, and Semen coicis Lachryma-jobi; said drug also containing an optional pharmaceutically acceptable carrier.

Embodiment 2. The use according to Embodiment 1 wherein said herbal composition comprises components having following weight ratios: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryma-jobi, 15-21% w/w.

Embodiment 3. The use according to Embodiment 2 wherein said herbal composition comprises components having following weight ratios: Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis, 18% w/w; Rhizoma Atractylodis Macrocephalae, 18% w/w; and Semen coicis Lachryma-jobi, 18% w/w.

Embodiment 4. The use according to Embodiment 1 wherein said herbal composition comprises components having following weight ratios: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryma-jobi, 15-21% w/w.

Embodiment 5. The use according to Embodiment 4 wherein said herbal composition comprises components having following weight ratios: Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis, 18% w/w; Rhizoma Atractylodis Macrocephalae, 18% w/w; and Semen coicis Lachryma-jobi, 18% w/w.

Embodiment 6. The use according to any one of Embodiments 1-5 wherein said disease is IBD.

Embodiment 7. The use according to Embodiment 6 wherein said IBD includes ulcerative colitis and Crohn's disease; or said IBD is ulcerative colitis.

Implementation plan 8. According to the uses described in implementation plans 1-7, the drug is provided in the form of tablets, pills, drip pills, capsules, granules, powders, suppositories, powders, ointments, patches, injections, solutions, suspensions, sprays, washes, drops, rubs or emulsions.

Implementation plan 9. According to the use described in implementation plan 8, the drug is provided in the form of injections, solutions, suspensions, sprays, washes, drops, rubs or emulsions.

Implementation plan 10. According to the use described in implementation plan 9, the drug is provided at a concentration of about 0.09-0.18 mg/mL.

Implementation plan 11. According to any one of the uses described in implementation plans 1-10, the pharmaceutically acceptable carrier includes: saline solution, sugars, gelatin, starches and cellulose.

Implementation plan 12. According to any one of the uses described in implementation plans 1-11, the pH of the carrier is 3-11, 5-9 or 7-8.

Implementation plan 13. According to any one of the uses described in implementation plans 1-12, the drug is administered at a dose of 1-4 g/kg body weight or at a dose of 2 g/kg body weight.

Implementation plan 14. The use of herbal composition in preparing a drug for increasing the proportion of CD4⁺Foxp3⁺ T_{reg} cells in individuals in need thereof, wherein said herbal composition comprises the following components: Fructus Terminaliae Chebulae; Radix Paeoniae Lactiflorae; Cortex Magnoliae Officinalis; Rhizoma Corydalis Yanhusuo; Herba Polygoni Chinensis; Rhizoma Atractylodis Macrocephalae; and Semen coicis Lachryma-jobi.

Implementation plan 15. According to the use described in implementation plan 14, said herbal composition comprises components having the following weight ratios: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryma-jobi, 15-21% w/w.

Implementation plan 16. According to the use described in implementation plan 15, said herbal composition comprises components having the following weight ratios: Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis, 18% w/w; Rhizoma Atractylodis Macrocephalae, 18% w/w; and Semen coicis Lachryma-jobi, 18% w/w.

Implementation plan 17. According to the use described in implementation plan 14, said herbal composition is composed of components having the following weight ratios: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryma-jobi, 15-21% w/w.

Implementation plan 18. According to the use described in implementation plan 17, said herbal composition is composed of components having the following weight ratios: Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis, 18% w/w; Rhizoma Atractylodis Macrocephalae, 18% w/w; and Semen coicis Lachryma-jobi, 18% w/w.

Implementation plan 19. The use of herbal composition in preparing a drug for restoring intestinal microbiota composition and restoring intestinal AHR expression in individuals in need thereof wherein said herbal composition comprises the following components: Fructus Terminaliae Chebulae; Radix Paeoniae Lactiflorae; Cortex Magnoliae Officinalis; Rhizoma Corydalis Yanhusuo; Herba Polygoni Chinensis; Rhizoma Atractylodis Macrocephalae; and Semen coicis Lachryma-jobi. The herbal medicine formula also contains an optional pharmaceutically acceptable carrier.

Implementation plan 20. According to the use described in implementation plan 19 wherein said herbal composition comprises components having the following weight ratios: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryma-jobi, 15-21% w/w.

Implementation plan 21. According to the use described in implementation plan 20 wherein said herbal composition comprises components having the following weight ratios: Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis, 18% w/w; Rhizoma Atractylodis Macrocephalae, 18% w/w; and Semen coicis Lachryma-jobi, 18% w/w.

Implementation plan 22. According to the use described in implementation plan 19 wherein said herbal composition is composed of components having the following weight ratios: 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryma-jobi, 15-21% w/w.

Implementation plan 23. According to the use described in implementation plan 22 wherein said herbal composition is composed of components having the following weight ratios: Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis, 18% w/w; Rhizoma Atractylodis Macrocephalae, 18% w/w; and Semen coicis Lachryma-jobi, 18% w/w.

### EXAMPLES

This invention describes preferred embodiments, and those skilled in the art can make appropriate changes to the embodiments and examples described in this invention after reading this invention. Therefore, the content of this invention requested for protection includes all equivalent modifications and variations of the subject matter of the claims of this invention within the legally permissible scope.

Animals and Experimental Materials

All animal experiments were approved by the Ethics Committee of the University of Macau. C57BL/6J mice and Rag 1^{-/-} immunodeficient mice were purchased from Jackson Laboratories in the United States and bred in-house at the University of Macau's specific pathogen-free (SPF) animal center. One week prior to the experiment, mice were acclimated to the environment under a 12-hour dark/light cycle with free access to food and water, with temperature controlled between 22.5±0.5° C. IFLX was purchased from Schering-Plough (Kenilworth). DSS (M.W. 36,000-50,000, colitis grade) was obtained from MP Biomedicals (Santa Ana, CA USA). The herbal composition for JCM-16021 were provided by Peili (Nanning Pharmaceutical Co. Ltd (Nanning, China)). All other materials and reagents were purchased from Thermo Fisher Scientific and Sigma-Aldrich or otherwise stated.

Experimental Methods

The study was conducted at the University of Macau Animal Center, where animals were housed by the center.

Day one of the experiment is defined as i) adoptive transfer of neonatal CD4⁺ T cells to Rag 1^{-/-} mice or ii) first day of DSS administration. JCM-16021 was orally administered to mice (weighing approximately 22-24 grams) at doses of 1.0 and 2.0 g/kg. Body weight and fecal bleeding were monitored every three days. Mice were euthanized and serum, colon tissue, spleen, mesenteric lymph nodes and feces were collected for further analysis.

Fecal Occult Blood Test

Fecal occult blood test kit was purchased from Nanjing Jiancheng Bioengineering Institute and performed according to the kit instructions.

Histological staining, PAS staining, immunohistochemical staining and immunofluorescent staining:
At the end of the experiment, mouse colon tissue was preserved using 4% paraformaldehyde fixed at 4° C. for 2 hours and embedded in paraffin. After dewaxing, mouse colon tissue sections were washed with PBS and blocked with 3% H₂O₂. Tissue sections were then blocked with blocking solution (10% horse serum, 0.1% bovine serum albumin, 0.2% gelatin) at room temperature for 1 hour to block non-specific sites. Colon tissue was then incubated with different primary antibodies prepared with blocking solution including PCNA, CD44, Iba1, Claudin, p65, c-Rel and Ahr at 4° C. overnight followed by washing with PBS-T buffer and incubation with corresponding secondary antibodies. Sections not incubated with primary antibody were used as negative controls during analysis.

For immunohistochemical staining, HRP-conjugated secondary antibody signals were detected using DAB peroxidase substrate kit (Vector Laboratories, Burlingame, CA USA), counterstained with hematoxylin and fixed for immunohistochemical analysis.

For immunofluorescent staining, cell nuclei were observed using Hoechst 33342 and signals were measured using Leica TCS SP8 confocal microscope.

For H&E staining and PAS staining, slides were dewaxed and stained using commercially available kits from Nanjing Jiancheng Bioengineering Institute.

Ulcerative Colitis Pathological Index Scoring

Histological grading of colitis were analyzed based on inflammatory features, extent, regeneration, crypt damage and percentage in hematoxylin-eosin and periodic acid-Schiff stained colon sections. Colitis scores will be determined by two independent researchers blinded to animal treatment.

Real-Time Quantitative PCR (qRT-PCR)

Total RNA was extracted from tissues using Trizol reagent (Invitrogen) and reverse transcribed into cDNA using cDNA synthesis kit (Bio-Rad Laboratories). The cDNA was diluted 50-fold as a template for qRT-PCR. According to the kit instructions mRNA expression levels were detected using SYBR Green fluorescent probe. Primers used for qRT-PCR are: Tnf-α, forward primer 5′-GACGTGGAACTGGCAGAAGAG-3′ (SEQ ID NO:1), reverse primer 5′-TTGGTGGTTTGTGAGTGTGAG-3′ (SEQ ID NO:2); Mcp-1, forward primer 5′-GGCTCAGCCAGATGCAGTTA-3′ (SEQ ID NO:3), reverse primer 5′-GCTGCTGGTGATCCTCTTGT-3′ (SEQ ID NO:4); 18S rRNA, forward primer 5′-GTAACCCGTTGAACCCCATT-3′ (SEQ ID NO:5), reverse primer 5′-CCATCCAATCGGTAGTAGCG-3′ (SEQ ID NO:6).

Bead-Based Serum Cytokine Level Measurement

The levels of pro-inflammatory cytokines (#740150) and chemokines (#740007) in mouse serum were measured using LEGENDplex™ bead-based immunoassays (BioLegend, San Diego, CA, USA). Specific measurements were performed according to the product instructions.

Isolation of Colon Lamina Propria Lymphocytes

After dissecting and isolating the colon from mice, it was washed four times in ice-cold PBS (137 mM NaCl, 2.7 mM KCl, 10 mM Na₂HPO₄, 1.8 mM KH₂PO₄, pH 7.4) and then cut into approximately 1 cm long fragments. Intestinal epithelial cells and lamina propria were separated by vigorous shaking in HBSS buffer containing 2 mM EDTA at 37° C. for 30 minutes. Intestinal epithelial cells were collected by centrifugation at 1,000 rpm at 4° C. The remaining colon tissue was incubated with digestion buffer (DMEM medium, 5% fetal calf serum, 1 mg/mL collagenase IV and DNAse I) at 37° C. with shaking for 40 minutes to separate colon lamina propria lymphocytes. The digested colon tissue cell suspension was filtered through a 70 micron cell filter and centrifuged. The cells were then resuspended in a 40:80 Percoll gradient of 10 ml of 40% Percoll gradient and placed on top of a 5 mL of 80% Percoll gradient in a 15 mL Falcon tube. Colon lamina propria lymphocytes were separated by Percoll gradient centrifugation at room temperature at 1,800 rpm for 20 minutes. Colon lamina propria lymphocytes collected between the Percoll density gradients were washed with DMEM medium and used for subsequent flow cytometry analysis.

Flow Cytometry Analysis of Regulatory T Cells

Colon lamina propria lymphocytes were diluted to 1×10⁵ cells/mL and subjected to intracellular staining after fixation and permeabilization. Cells were stained with a primary antibody diluted 1:100 at 4° C. in the dark for 30 minutes and analyzed by flow cytometry using antibodies against CD3 (APC, BD Biosciences), CD4 (FITC, BD Biosciences), and Foxp3 (PE, BD Biosciences). Analysis was performed using a BD LSRFortessa™ flow cytometer (BD Biosciences) and data obtained was analyzed using FlowJo software.

Intestinal Microbiota 16S rRNA Sequencing

Bacterial DNA was extracted from mouse feces using phenol:chloroform:isoamyl alcohol (25:24:1, pH 7.9) and the conserved region of the 16S rRNA was amplified by PCR. V4 region of the bacterial genome was sequenced using PE250/PE300 dual-end sequencing platform according to Illumina MiSeq/Novaseq (Illumina, San Diego, CA USA) recommendations. Data analysis was performed by GENEWIZ (Suzhou, China) using QIIME software package.

Statistical Analysis

No animal data was excluded from analysis. All data obtained will be expressed as mean±standard error of the mean (SEM). All experiments were repeated at least three or more times and intergroup differences in measured variables were analyzed using one-way or two-way ANOVA methods using GraphPad Prism 5.0 software. Results were considered statistically significant when p<0.05.

### Example 1: Preparation of a Drug Containing Herbal Composition JCM-16021

Two grams of JCM-16021 dry powder containing 10% starch shaping agent (JCM-16021 composition: Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis, 18% w/w; Rhizoma Atractylodis Macrocephalae, 18% w/w; Semen coicis Lachryma-jobi, 18% w/w was added to 2 grams of Solutol® polyethylene glycol (15)-hydroxystearate and incubated at 60° C. for 30 minutes, then brought to a final volume of 10 mL with distilled water. The supernatant was separated from the debris by centrifugation at room temperature (25° C.) at 12,000 rpm for 10 minutes. Based on the dry weight of the dry powder, the concentration of JCM-16021 was calculated to be 0.18 mg/mL JCM-16021 (2 g/10 mL×0.9). The herbal extract was stored at 4° C. and used within 24 hours. Any residual sediment formed upon standing was vortexed into suspension and used to treat animals.

### Example 2: Therapeutic Effect of Herbal Composition JCM-16021 on CD4⁺ Naive T Cell Transfer-Induced Ulcerative Colitis

CD4⁺ Naive T Cell Transfer-Induced Ulcerative Colitis Model

CD4⁺ naive T cells were isolated and purified from the spleens of wild-type C57BL/6J mice using a naive CD4+ T cell isolation kit (#130-104-453, Miltenyi Biotech) according to the manufacturer's recommendations. The isolated CD4⁺ naive T cells were incubated with antibodies against CD4 (#100406, BioLegend), CD25 (#17025182, eBiosciences) and CD45RB (#103314, BioLegend) and purity was analyzed by BD Fortessa flow cytometer (BD Biosciences). 4×10⁵ CD4⁺ naive T cells were intraperitoneally injected into Rag 1^{-/-} immunodeficient mice. Clinical symptoms of colitis including fecal occult blood, loose stool/diarrhea, rough/hunched posture and weight loss were monitored every three days thereafter. Any mouse with a weight loss exceeding 20% of its initial weight or exhibiting severe signs of illness was euthanized. JCM-16021 treatment was initiated three weeks after naive T cell transfer with twice daily oral gavage (doses of 1.0 and 2.0 g/kg body weight) for three weeks. IFLX was administered intraperitoneally at a dose of 5 mg/kg as a positive control drug on the first day of JCM-16021 administration once weekly for three weeks.

Experimental Results

JCM-16021 Exhibits Significant Therapeutic Effect in Naive CD4⁺ T Cell Transfer-Induced Ulcerative Colitis Mice

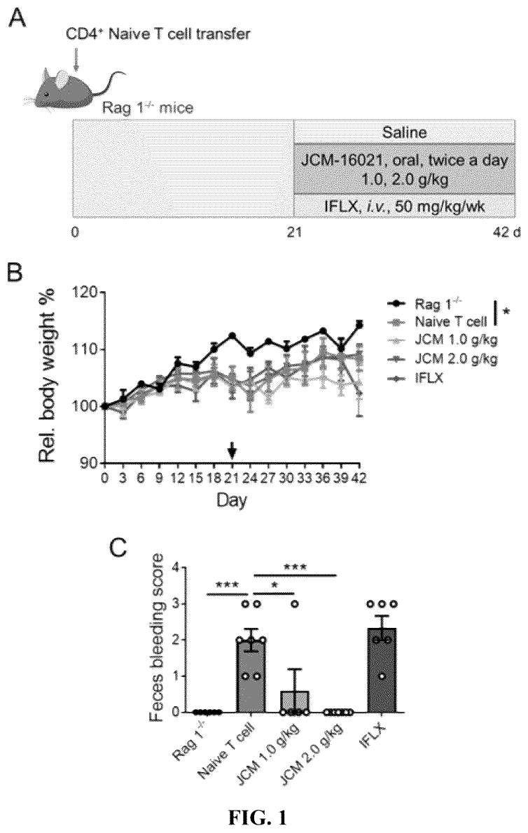

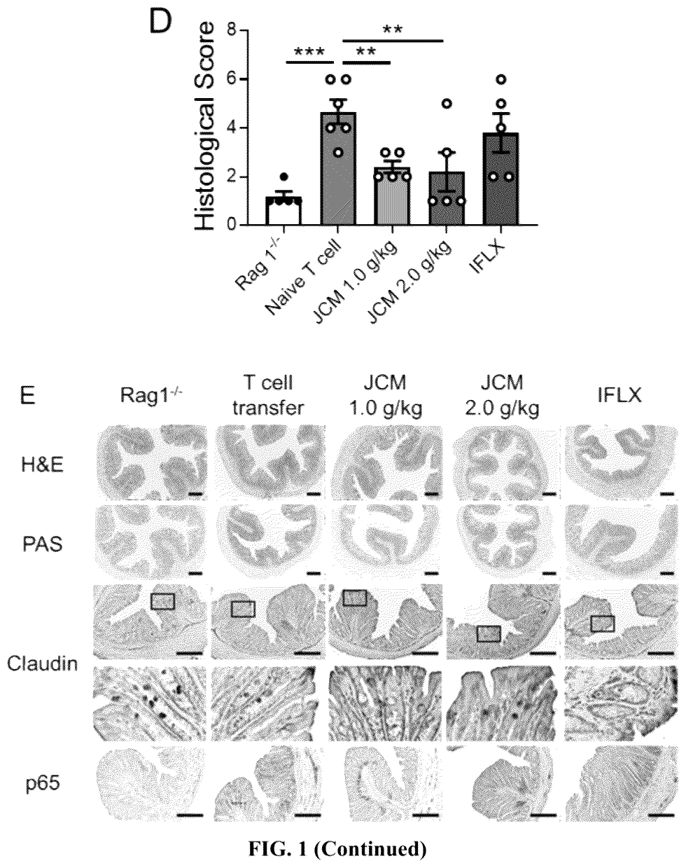

To determine the efficacy of JCM-16021 in naive T cell transfer-induced colitis in Rag 1^{-/-} immunodeficient mouse model, purified CD4⁺CD25⁻CD45RB^{high} naive T cells were intraperitoneally injected into Rag 1^{-/-} immunodeficient mice (FIG. 1A). JCM-16021 was orally administered at doses of 1.0 g/kg and 2.0 g/kg body weight three weeks after naive T cell transfer (FIG. 1B). IFLX was used as a positive control drug (FIG. 1). Compared to sham-operated Rag 1^{-/-} mice, mice in the CD4⁺ naive T cell transfer group showed significant weight loss (FIG. 1B). JCM-16021 treatment dose-dependently reduced fecal occult blood (FIG. 1C) and ulcerative colitis pathological index (FIG. 1D); whereas IFLX treatment did not exhibit similar activity. Hematoxylin-eosin (H&E) and periodic acid-Schiff (PAS) staining results showed that colon tissue morphology in mice receiving CD4⁺ naive T cell transplantation was abnormal with significant epithelial cell damage and abnormal mucin (FIG. 1E). In contrast, mice treated with JCM-16021 at a dose of 2.0 g/kg exhibited normal epithelial barrier and mucin similar to Rag 1^{-/-} mice (FIG. 1E). Suppressed colon Claudin expression levels in mice receiving CD4⁺ naive T cell transfer were restored by JCM-16021 treatment but not in infliximab-treated mice (FIG. 1E). JCM-16021 also significantly reduced high levels of p65 expression in colitis mice (FIG. 1E). JCM-16021 at a dose of 2.0 g/kg exhibited better therapeutic efficacy than at a dose of 1.0 g/kg; whereas under the same experimental conditions, infliximab was less effective than JCM-16021.

JCM-16021 Treatment Inhibits Intestinal Inflammation in Ulcerative Colitis Mice

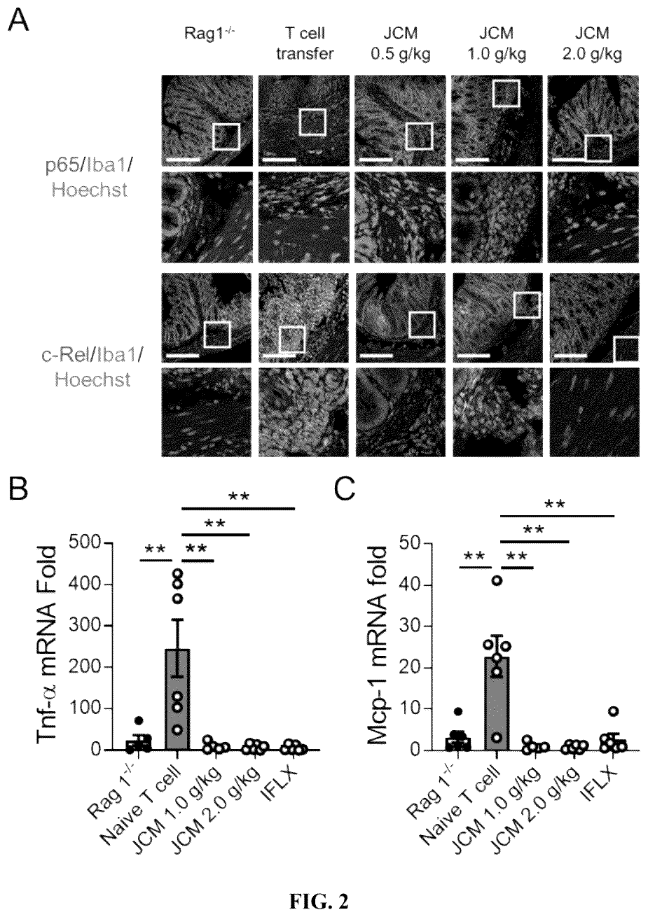

Immunofluorescent staining results showed that colon Iba 1-positive macrophages in naive CD4⁺ T cell transfer-induced ulcerative colitis mice exhibited significant nuclear localization of NF-κB p65 and c-Rel (FIG. 2A). Specifically, activation of p65 and c-Rel co-localized with ionized calcium-binding adapter molecule 1 (Iba 1), a macrophage-specific calcium-binding protein. The inhibitory effect of JCM-16021 on NF-κB p65 nuclear translocation and c-Rel activation was dose-dependent, indicating that colon-infiltrating macrophages were also effectively inhibited in CD4⁺ naive T cell transfer-induced colitis. Both JCM-16021 and IFLX treatment significantly reduced the expression levels of pro-inflammatory cytokine Tnf-α and chemokine Mcp-1 mRNA in the colon tissue of colitis mice (FIGS. 2B-2C).

JCM-16021 Treatment Induces Proliferation of Crypt Region Stem Cells, Expression of AHR and Increased Proportion of Regulatory T Cells

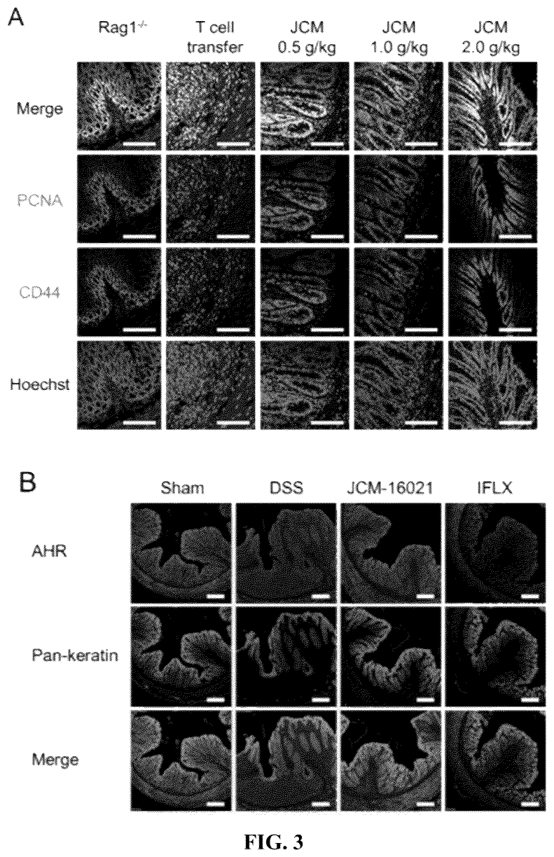

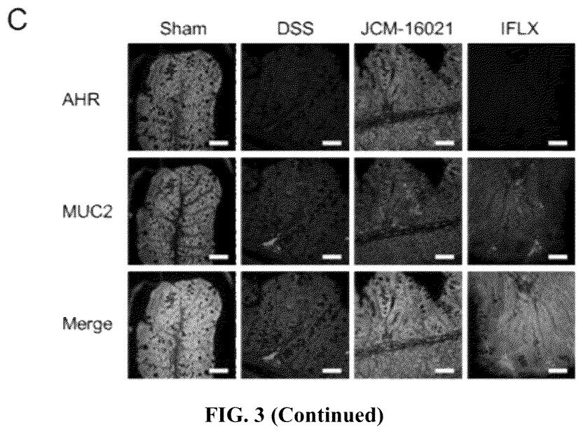

Proliferation of colon crypt stem cells is critical for the restoration of the intestinal epithelial barrier. In CD4⁺ naive T cell transfer-induced colitis mice, the expression level of PCNA in CD44⁺ stem cells was significantly reduced, while after JCM-16021 treatment, the expression level of PCNA in CD44⁺ stem cells was restored to the same level as normal crypts in a dose-dependent manner (FIG. 3A).

Immunofluorescent staining results showed that the defective expression of AHR and MUC2 in CD4⁺ naïve T cell-transfer induced colitis mice was elevated with JCM-16021 at 2.0 g/kg back to the level comparable to that in sham mice (FIG. 3B-3C). The expression of AHR co-localized with the intestinal epithelial marker pan-keratin (FIG. 3B). The expression of MUC2 Also Co-Localized with AHR Positive Cells in the Large Intestine (FIG. 3C).

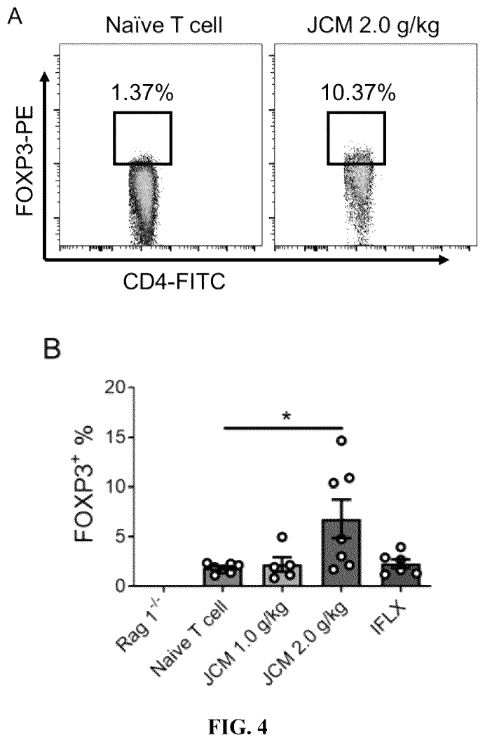

The balance of proportion and number of regulatory T cells is critical for the treatment of inflammatory diseases, especially for restoring intestinal homeostasis in ulcerative colitis. The inventors analyzed and quantitated the ratio of T_{reg} cells using FACS analysis. The representative gating strategy is shown in FIG. 4A. The inventors identified that the ratio of CD4₊Foxp3₊ T_{reg} cells in the isolated LPMC was significantly increased with JCM-16021 treatment at 2.0 g/kg using FACS analysis (FIG. 4B-4D). In contrast, the ratio of CD4⁺Foxp3⁺ T_{reg} cells was similar in CD4⁺ naïve T cell transfer-induced colitis mice and mice that received IFLX treatment (FIG. 4B).

JCM-16021 Treatment Reshapes the Gut Microbiota of Mice

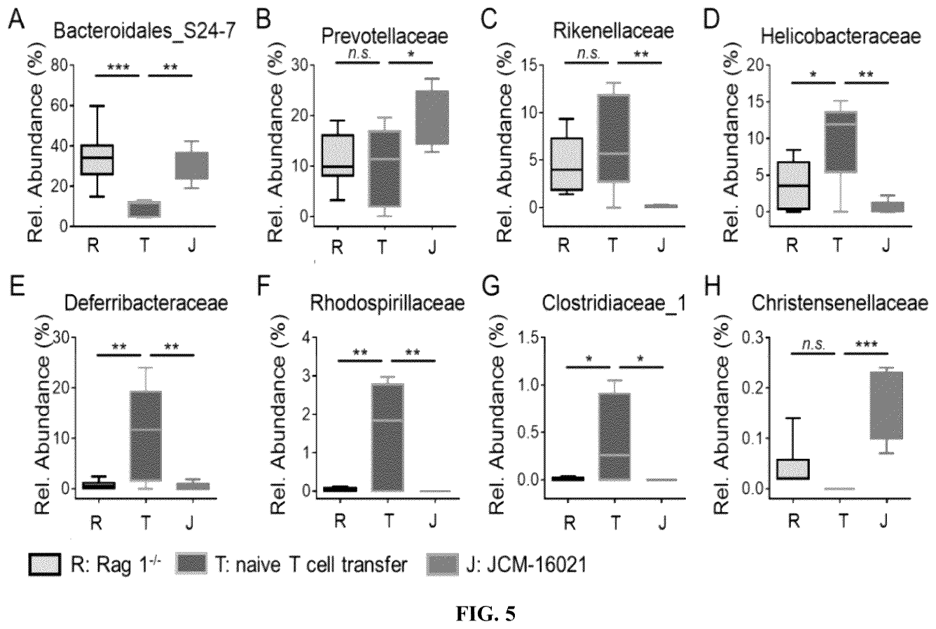

The gut microbiota plays an important role in the pathogenesis and treatment mechanisms of inflammatory bowel diseases such as ulcerative colitis. We used 16S rRNA gene sequencing to analyze the composition of the fecal microbiota of mice after JCM-16021 treatment. All mouse feces were immediately frozen in liquid nitrogen after collection. Sequencing results showed that at a dose of 2.0 g/kg, the gut microbiota of mice treated with JCM-16021 was significantly different from that of ulcerative colitis mice induced by CD4⁺ naive T cell transfer at the species level, but very close to that of sham-operated mice (FIG. 5). Among the eight most significantly changed bacterial species, three species recovered to the same level as sham-operated mice after JCM-16021 treatment, including Bacteroidales S24-7, Prevotellaceae, and Christensenellaceae (FIG. 5); while five species that were significantly increased in colitis mice were significantly reduced to sham-operated levels after JCM-16021 treatment, including Rikenellaceae, Helicobacteraceae, Deferribacteraceae, Rhodospirillaceae, and Clostridiaceae_1 (FIG. 5).

### Example 3: Therapeutic Effect of Herbal Composition JCM-16021 in Dextran Sulfate Sodium (DSS) Induced Ulcerative Colitis Mice

Dextran Sulfate Sodium (DSS) Induced Ulcerative Colitis Mouse Model

Male and female C57BL/6J mice aged 6-8 weeks were given 2.5% dextran sulfate sodium salt for 5 consecutive days followed by recovery with water for 2 cycles. In long-term drug treatment experiments, 2% DSS was used for four consecutive weeks.

Experimental Results

JCM-16021 Exhibits Excellent Therapeutic Effects in DSS Induced Ulcerative Colitis Mice

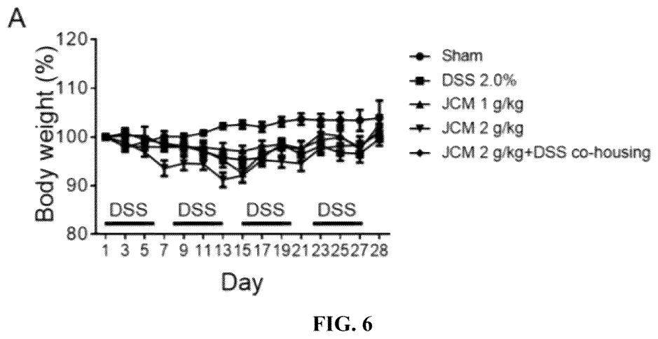

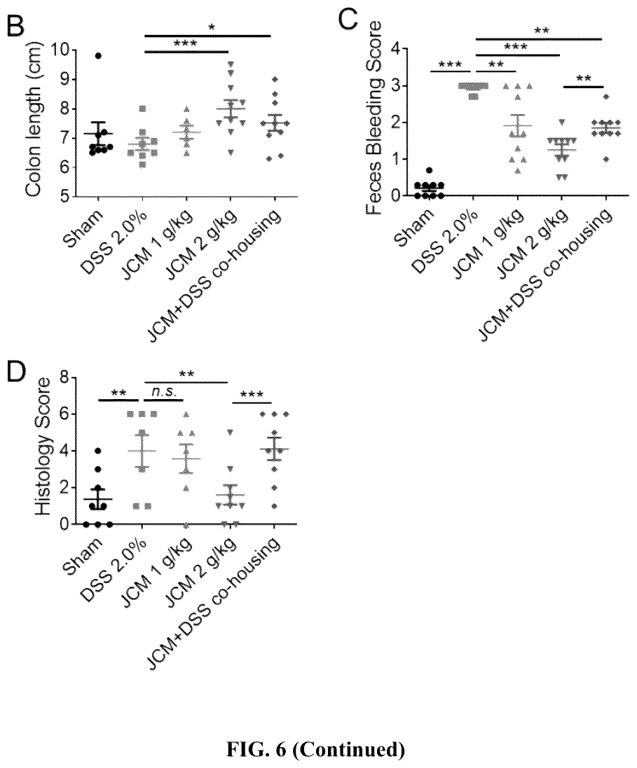

We further tested the therapeutic effect of JCM-16021 in dextran sulfate sodium-induced ulcerative colitis mice (FIG. 6). Although there was no change in body weight in all groups of long-term dextran sulfate sodium-induced ulcerative colitis mice throughout the treatment period (FIG. 6A), JCM-16021 dose-dependently reversed dextran sulfate sodium-induced colon shortening (FIG. 6B), reduced fecal occult blood index (FIG. 6C), and ulcerative colitis pathological index (FIG. 6D). This result is consistent with the efficacy in the CD4⁺ naive T cell transfer-induced mouse colitis model. In addition, after co-housing with untreated colitis mice, co-housing significantly reduced the therapeutic effect of JCM-16021 at a dose of 2.0 g/kg (FIGS. 6B-6D).

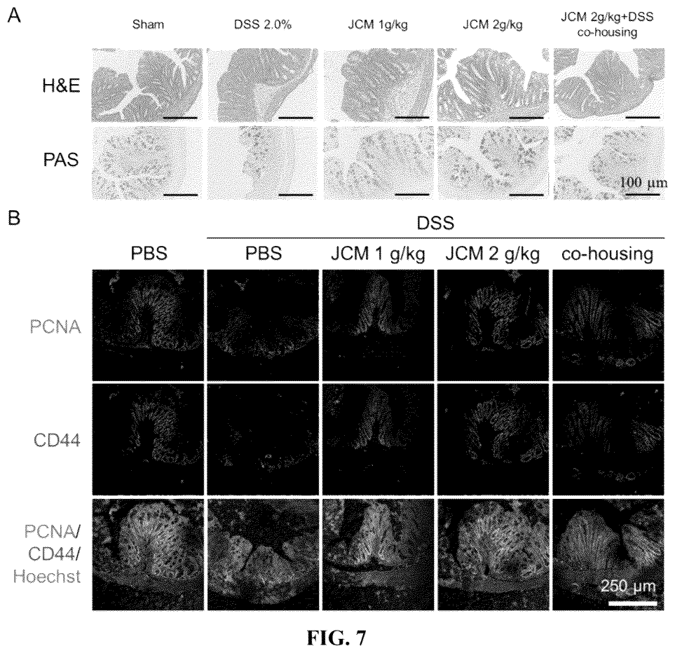

Hematoxylin-eosin and periodic acid-Schiff staining showed that colon tissue from dextran sulfate sodium-induced ulcerative colitis mice exhibited abnormal pathological features including loss of intestinal epithelial integrity, thickening of the lamina propria, and infiltration of immune cells (FIG. 7A). After JCM-16021 treatment, these pathological features of colitis were dose-dependently restored to sham-operated mouse levels (FIG. 7A). PCNA expression in colon crypt stem cells of JCM-16021-treated mice also gradually increased with dose to levels similar to those of sham-operated mice (FIG. 7B). However, after co-housing with colitis mice, these therapeutic effects were similar to those of untreated colitis mice (FIG. 7).

JCM-16021 Treatment Reduces Systemic Inflammation

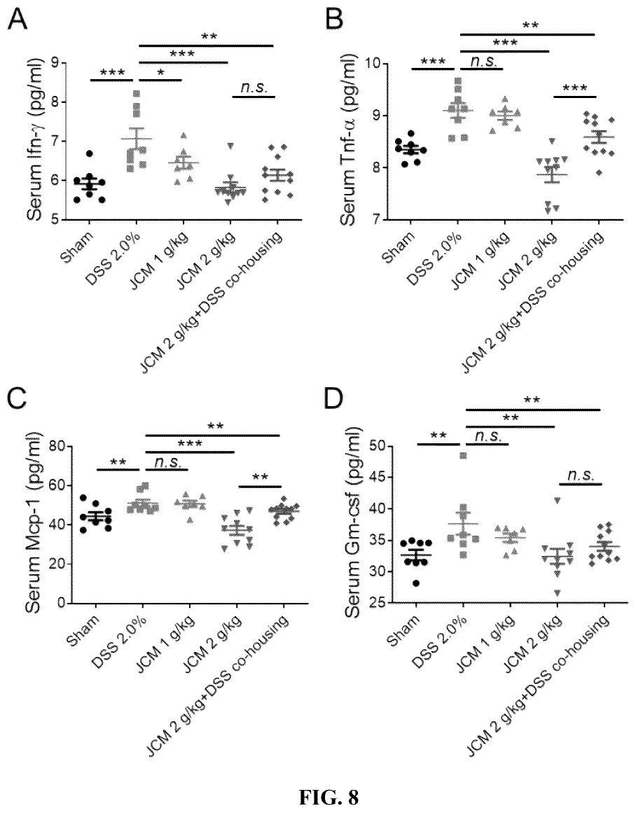

After JCM-16021 treatment, systemic inflammation markers in dextran sulfate sodium-induced ulcerative colitis mice, including pro-inflammatory cytokines Ifn-γ and Tnf-α and chemokines Mcp-1 and Gm-csf in serum, were significantly reduced with increasing doses of JCM-16021 (FIG. 8). Among them, the inhibitory effect of JCM-16021 on Tnf-α and Mcp-1 in serum of colitis mice disappeared after co-housing with colitis mice; while Ifn-γ and Gm-csf serum levels were independent of whether or not they were co-housed with colitis mice (FIG. 8).

JCM-16021 Restored Intestinal Expression of AHR and MUC2 and Increased the Ratio of T_{reg} Cells in the DSS Murine Colitis Model

The inventors examined the intestinal expression of AHR and MUC2 in the DSS murine colitis model with JCM-16021 treatment. Immunofluorescent staining results showed that the intestinal expression levels of AHR and MUC2 were markedly decreased in DSS colitis mice but were restored with JCM-16021 treatment (FIGS. 9A and 9B). The expression of AHR co-localized with the intestinal epithelial cell marker pan-keratin, while the expression of MUC2 co-localized with AHR in the colon sections (FIGS. 9A and 9B).

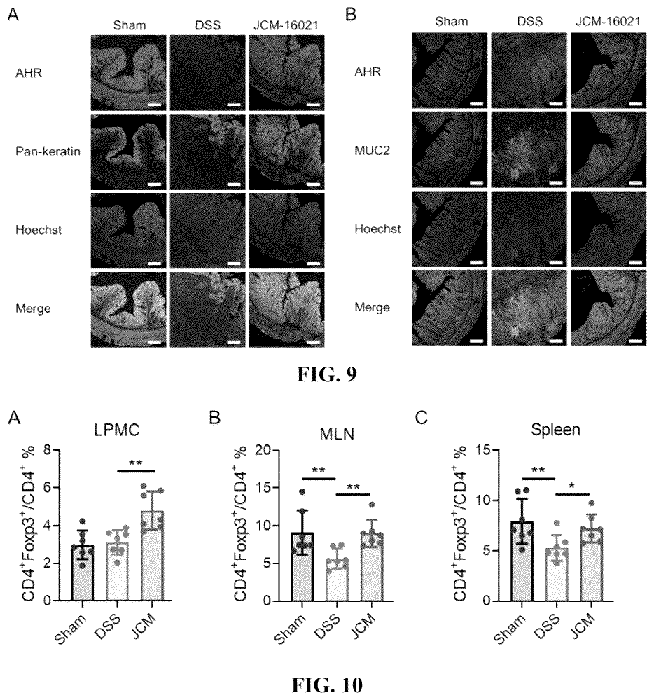

The inventors analyzed and quantitated the ratio of T_{reg} cells in JCM-16021-treated colitis mice using FACS analysis. The inventors found that the ratio of CD4⁺Foxp3⁺ T_{reg} cells in the isolated LPMC, MLN, and spleen was significantly increased in mice that received JCM-16021 treatment at 2.0 g/kg compared to those from the DSS model group (FIG. 10A-10C). The ratio of CD4⁺Foxp3⁺ T_{reg} cells was similar in mice from the sham group to the DSS-induced colitis group in the LPMC (FIG. 10A), while it was markedly decreased in MLN and spleen in mice with DSS-induced colitis compared to those from the sham group (FIG. 10B-10C).

JCM-16021 Treatment Reshapes Gut Microbiota in Dextran Sulfate Sodium-Induced Ulcerative Colitis Mice

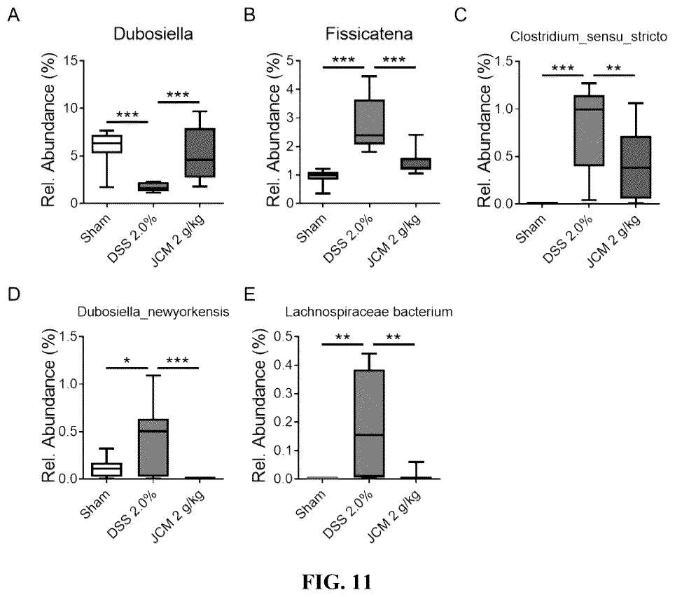

To analyze whether JCM-16021 treatment could change the composition of the gut microbiota, we collected intestinal contents from sham-operated, dextran sulfate sodium-induced colitis and JCM-16021-treated groups of mice. Using 16S rRNA gene sequencing to determine specific changes in gut microbiota. Sequencing results showed that after JCM-16021 treatment, the gut microbiota composition of dextran sulfate sodium-induced colitis mice recovered to a level comparable to that of sham-operated mice (FIG. 9), with changes in bacterial species mainly including Dubosiella, Fissicatena, Clostridium_sensu_stricto, Dubosiella_newyorknesis and Lachnospiraceae bacterium (FIG. 11).

Conclusion: The present invention provides an herbal composition for the treatment of IBD, especially ulcerative colitis. The present invention also provides a method for improving colon-intestinal barrier repair by promoting the proliferation of colon crypt zone stem cells. The present invention improves the treatment of the disease by increasing the proportion of CD4⁺Foxp3⁺ regulatory T (T_{reg}) cells in the lamina propria lymphocytes. The present invention also provides a therapeutic method for restoring the composition of the gut microbiota and the intestinal expression of the aryl hydrocarbon receptor. In addition, the present invention provides a drug combination with therapeutic effects superior to those of infliximab, a biologic agent used clinically.

What is claimed is:

1. A method of treating a disease by increasing regulatory T cells in a subject in need thereof, the method comprising: administering a therapeutically effective amount of a herbal composition comprising: Fructus Terminaliae Chebulae, Radix Paeoniae Lactiflorae, Cortex Magnoliae Officinalis, Rhizoma Corydalis Yanhusuo, Herba Polygoni Chinensis, Rhizoma Atractylodis Macrocephalae, and Semen coicis Lachryrna-jobi; and optionally a pharmaceutically acceptable carrier.

2. The method of claim 1, wherein the herbal composition comprises: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryrna-jobi, 15-21% w/w.

3. The method of claim 2, wherein the herbal composition comprises: Fructus Terminaliae Chebulae, 9% w/w; Radix Paeoniae Lactiflorae, 14% w/w; Cortex Magnoliae Officinalis, 9% w/w; Rhizoma Corydalis Yanhusuo, 14% w/w; Herba Polygoni Chinensis, 18% w/w; Rhizoma Atractylodis Macrocephalae, 18% w/w; and Semen coicis Lachryrna-jobi, 18% w/w.

4. The method of claim 1, wherein the disease is inflammatory bowel disease.

5. The method of claim 4, wherein the inflammatory bowel disease is ulcerative colitis or Crohn's disease.

6. The method of claim 1, wherein the herbal composition is provided in the form of a tablet, a pill, a drop, a capsule, granules, a powder, a suppository, powders for dispersion in water or other liquid for oral administration or for external application to skin or mucous membranes, a patch, an injection, a solution, a suspension, a spray, a wash, a drop, a rub, or an emulsion.

7. The method of claim 6, wherein the herbal composition is provided in the form of an injection, a solution, a suspension, a spray, a wash, a drop, a rub, or an emulsion.

8. The method of claim 7, wherein the herbal composition is provided at a concentration of about 0.09-0.18 mg/mL.

9. The method of claim 1, wherein the pharmaceutically acceptable carrier comprises: a saline solution, a sugar, a gelatin, starch, Ringer's solution, or cellulose.

10. The method of claim 1, wherein the pH of the pharmaceutically acceptable carrier is 3-11, 5-9, or 7-8.

11. The method of claim 1, wherein the herbal composition is administered at a dose of 1-4 g/kg body weight or 2 g/kg body weight.

12. A method for preparing a drug composition comprising an herbal composition, the method comprising: grinding the herbs and an optional pharmaceutically acceptable carrier into a dry powder; adding the dry powder to a water-soluble solvent and incubating at 60° C. for 30 minutes; adding distilled water; and separating the supernatant from debris, wherein the herbal composition comprises:
Fructus Terminaliae Chebulae, about 6-12% w/w;
Radix Paeoniae Lactiflorae, about 11-17% w/w;
Cortex Magnoliae Officinalis, about 6-12% w/w;
Rhizoma Corydalis Yanhusuo, about 11-17% w/w;
Herba Polygoni Chinensis, about 15-21% w/w;
Rhizoma Atractylodis Macrocephalae, about 15-21% w/w; and
Semen coicis Lachryrna-jobi, about 15-21% w/w.

13. An herbal composition comprising: Fructus Terminaliae Chebulae, 6-12% w/w; Radix Paeoniae Lactiflorae, 11-17% w/w; Cortex Magnoliae Officinalis, 6-12% w/w; Rhizoma Corydalis Yanhusuo, 11-17% w/w; Herba Polygoni Chinensis, 15-21% w/w; Rhizoma Atractylodis Macrocephalae, 15-21% w/w; and Semen coicis Lachryrna-jobi, 15-21% w/w.

14. A drug composition comprising an herbal composition of claim 13 and a pharmaceutically acceptable carrier.

15. The drug composition of claim 14, wherein the herbal composition is prepared by grinding the herbs and an optional pharmaceutically acceptable carrier into a dry powder; adding the dry powder to a water-soluble solvent and incubating at 60° C. for 30 minutes; adding distilled water; centrifuging at room temperature at a speed of from 7,500-12,000 rpm for from 10 minutes and separating the supernatant from debris.

16. The drug composition of claim 15, wherein the water-soluble solvent comprises polyethylene glycol(15)-hydroxystearate ester.

17. The drug composition of claim 14, wherein the herbal composition is provided in the form of a tablet, a pill, a drop, a capsule, a granule, a powder, a suppository, a powder for dispersion in water or other liquid for oral administration or for external application to skin or mucous membranes, a patch, an injection, a solution, a suspension, a spray, a wash, a drop, a rub, an emulsion.

18. The drug composition of claim 14, wherein the drug composition is provided in the form of an injection, a solution, a suspension, a sprays, a wash, a drop, a rub, or an emulsion.

19. The drug composition of claim 18, wherein the drug composition is provided at a concentration of about 0.09-0.18 mg/mL.

20. The drug composition of claim 18, wherein the drug composition is provided at a concentration of about 0.18 mg/mL.

21. The drug composition of claim 14, wherein the pH of the carrier is 3-11, 5-9, or 7-8.


SEQUENCE LISTING (6 sequences)

SEQ ID NO: 1 - DNA, 21 bp, Homo sapiens
  source 1..21: mol_type = genomic DNA
gacgtggaactggcagaagag

SEQ ID NO: 2 - DNA, 21 bp, Homo sapiens
  source 1..21: mol_type = genomic DNA
ttggtggtttgtgagtgtgag

SEQ ID NO: 3 - DNA, 20 bp, Homo sapiens
  source 1..20: mol_type = genomic DNA
ggctcagccagatgcagtta

SEQ ID NO: 4 - DNA, 20 bp, Homo sapiens
  source 1..20: mol_type = genomic DNA
gctgctggtgatcctcttgt

SEQ ID NO: 5 - DNA, 20 bp, Homo sapiens
  source 1..20: mol_type = genomic DNA
gtaacccgttgaaccccatt

SEQ ID NO: 6 - DNA, 20 bp, Homo sapiens
  source 1..20: mol_type = genomic DNA
ccatccaatcggtagtagcg